(19) United States
(12) Patent Application Publication
(10) Pub. No.: US 2009/0197299 A1
(43) Pub. Date: Aug. 6, 2009

(54) Method for Assessing Airborn Microorganisms

(75) Inventors: Diego Vargas, Lansdale, PA (US); Luis A. Baez, Simi Valley, CA (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 12/085,738

(22) PCT Filed: Nov. 29, 2006
(86) PCT No.: PCT/US2006/045613

§ 371 Date: May 29, 2008

Related U.S. Application Data
(60) Provisional application No. 60/741,811, filed on Dec. 2, 2005.

(51) Int. Cl. C12Q 1/06; C12Q 1/04

(52) U.S. Cl. 435/39; 435/34

(57) ABSTRACT

The present invention relates to an environmental sampling method for assessing airborne microorganisms. The method comprises retaining organisms suspended in air to a polymeric pad via gravitational settling or forced impact, dissolving the polymeric pad in a buffered salt solution, and determining the number and kind of microorganisms in the buffered salt solution. Neither the polymeric pad nor the buffered salt solution inhibits the growth of the microorganisms to be determined.

### BACKGROUND OF THE INVENTION

There is a tremendous need to quantify airborne microorganisms in the commercial microbiology industry (biotechnology). The enumeration of airborne microorganisms is also important for the evaluation of indoor air quality, infectious disease outbreaks, and agriculture health care investigation. (Jensen, et al, Am Ind Hyg Assoc J 53:660-7 (1992); Juozaitis, et al, Appl environ Microbiol, 60:861-870 (1994); Nevalainen, Atmospheric Environment, 26A:531-540 (1992)) Moreover, after the outbreak of Bacillus anthrax in the US Postal Service, there is an increased need in assessing occupational exposure to bio-aerosols.

Organisms suspended in air are sampled traditionally via microbiological settling plates with tryptic soy agar (TSA) or any other media or via impingement methods. The commonly used commercial environmental samplers include the Anderson-six-stage particle sizing sampler (6-STG), Ace Glass all-glass impinger-30 (AGI-30), Biotest Reuter centrifugal air sampler, etc. (ibid, Cohen, B. S. and S. V. Hering. 1995. Air Sampling Instruments for Evaluation of Atmospheric Contaminants, 8^{th} Ed. Cincinnati, Ohio: American Conference of Governmental Industrial Hygienists; Baron, Paul and Willeke, Klaus. 2001. Aerosol Measurement: principles, techniques and applications. 2^{nd} Ed.) The previously mentioned air sampler utilizes TSA as sampling medium which will become quickly saturated with a high concentration of organisms (except for the AGI-30).

Sampling organisms from the environment with settling plates is limited to sample concentration lower than 100 CFU/m³ in order to obtain colonies within plate count range. Impingement method allows sampling higher concentration, however there is a degree of cell injury caused by cell impact, therefore loss of cell viability. There is a need for a method that is capable of measuring any concentration of airborne microorganisms via plate settling or filtration without any cell injury.

The references cited herein are not admitted to be prior art to the claimed invention.

### SUMMARY OF THE INVENTION

The present invention relates to an environmental sampling method for assessing airborne microorganisms; the method comprises retaining organisms suspended in air to a polymeric pad via gravitational settling or forced impact, dissolving the polymeric pad in a buffered salt solution, and determining the number and kind of microorganisms in the buffered salt solution; neither the polymeric pad nor the buffered salt solution inhibits the growth of the microorganisms to be determined.

According to an embodiment of the present invention, the polymeric pad is preferably porous. More preferably, the polymeric pad is a calcium alginate pad.

The buffered salt solution is preferably a buffered sodium solution. More preferably, the buffered sodium solution is a sodium citrate solution with the pH at about 7.

The microorganisms to be determined can be bacteria, yeast, or filamentous fungi. According to an embodiment of the present invention, the microorganisms comprise B. diminuta. According to another embodiment of the present invention, the microorganisms comprise P. aeruginosa. According to a further embodiment of the present invention, the microorganisms comprise B. atrophaeus.

The step of determining can be achieved by diluting the buffer, pouring the diluted buffer solution on growth media, incubating the plates in the conditions suitable for the growth of the organisms to be determined, and counting the colonies.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### DETAILED DESCRIPTION OF THE INVENTION

The present invention relates to an environmental sampling method for recovering organisms suspended in the air, such as bacteria, yeast, and mold. The method comprises retaining organisms suspended in air to a polymeric pad via gravitational settling or forced impact, dissolving the polymeric pad in a buffered salt solution, and determining the number and kind of microorganisms in the buffered salt solution Neither the polymeric pad nor the buffered salt solution inhibits the growth of the microorganisms to be determined.

In the case of “forced impact”, a directional gas flow through the polymeric pad is created with the intent to promote impaction and adhesion (retention/entrapment) of an organism suspended in a gaseous medium. The stream can be created from a vacuum source on the opposite side of the gas to be sampled and separated by the polymeric pad. In this arrangement, a solid support to the filter pad may be required in order to maintain its shape (solid wire mesh support in a filter holder for example). Its main difference from “gravitational settling” can be found in that the former applies dynamic method to “drive” a volume of sampled atmosphere (gas) through the polymeric pad whereas the latter is more of a passive method for sampling where the organism settles down to the pad due to its own mass. In force impact you could measure an organism concentration per volume of gas sampled over time per area of polymeric pad (volumetric measurement), whereas in the gravitational settling you can measure organism concentration sampled over time per area of polymeric pad (surface area). Filtration will be pretty much the same as forced impaction since a stream is applied. In the case of filtration, retention by sieving or size exclusion also applies in addition to forced impaction where an organism can be retained by adhesion. In the case of the polymeric pad and different from a filtration device, there is no pore size rating for the polymeric mesh.

### 1. The Polymeric Pad and the Dissolving Solution

The polymeric pad is a pad made of polymer such as calcium alginate. It should be soluble in a buffered solution. On the other hand, the polymeric pad is preferably insoluble in water, so that it can be moistened to better retain the airborne organisms. The pad is preferably porous.

The polymeric pad is preferably made of calcium alginate. Calcium alginate is made from algin in the cell walls of marine brown algae. Algin is composed of alginic acid and its salts. Alginic acid is a linear polysaccharide made from two monomer substrates: mannuronate and guluronate. Sodium alginate extracted from brown algae is soluble and forms viscous solution. When calcium salt is added, the calcium-sodium ions exchange leads to the precipitation of insoluble calcium alginate. Textile fibers of calcium alginate can then be produced via rinsing and dehydration (Schenck, Wundforum online, http://www.hartmann-online.de/english/produkte/wundbehandlung/wundforum/default.htm; http://www.ispcorp.com/ptoducts/food/contet/brochure/alginates/reaction.html)

Calcium alginate is the “ideal dressing material” for moist wound treatment. (ibid.) Calcium alginate is porous biodegradable polymer that is able to dissolve in a solution of sodium salt, and can be used to make surgical pads. When applied to a wound, the dry fibers of calcium alginate absorb the exudate. The insoluble calcium alginate becomes soluble sodium alginate because of the reverse ion exchange. The calcium ions are exchanged for sodium ions in the blood and wound exudate. The fibers of calcium alginate then gradually turn into moist gel that fills and securely covers the wound. (ibid.)

According to an embodiment of the present invention, calcium alginate pad is an excellent material to be used as sampler of organisms suspended in air via gravitational deposition or via filtration in environmental microbiology. It also permits enumeration of high concentration of airborne microorganisms. The calcium alginate pad can easily be dissolved in an appropriate buffer, providing the gentle conditions to the organisms, therefore minimizing cell injury and loss of cell viability.

Examples of calcium alginate pads that are commercially available include Algosteril®, Comfeel® Alginate, Kaltostat®, and Sorbsan® (Agren, Br J Plast Surg March, 49:129-134 (1996)), Curasorb™, Algisite®, CalciCare™, and Kalginate® (http://woundcareshop.safeshopper.com/5/cat5.htm) According to a preferred embodiment of the present invention, the calcium alginate pad is the Kalginate® surgical pad for wound dressing manufactured by DeRoyal Wound Care. The calcium alginate pads dissolve very easily in sodium citrate solutions through the mechanism of ionic exchange. These pads, once moistened, provide a suitable environment for the airborne microorganism to adhere to it.

According to an embodiment of the present invention, a calcium alginate pad is used to collect environmental samples of microorganisms, such as Bacillus atrophaeus or Brevundimonas diminuta, via filtration or by settling plate methodology. The calcium alginate pad is then dissolved in sodium citrate buffer. The enumeration of the collected microorganisms is performed via normal serial dilution.

Thanks to the solubility of calcium alginate in the solution of sodium salt, the present invention can be used to measure concentration of environmental organisms equal or greater than 10⁶ Colony Forming Units per Liter suspended in air. The polymer easily dissolves in sodium salts via ionic exchange, therefore the polymer matrix can retain organisms and particles via deposition or filtration for subsequent quantification at any level. The calcium alginate pads can then be dissolved in an appropriate buffered salt solution, such as sodium citrate, followed by serial dilution and plating.

Colonies can then be counted, using the standard plate count methods after the concentration is diluted to less than 300 CFU per ml. The polymeric matrix was able to yield 100% recovery from organism suspension at a concentration greater than 10⁸ CFU/mL and above 10⁶ CFU/L from air samples.

The dissolving solution should not inhibit the growth of microorganisms to be assessed. The dissolving solution is preferably a solution of sodium salts, more preferably a solution of sodium citrate. Thy dissolving solution preferably has a pH at about 7.

### 2. The Microorganisms to be Assessed

The microorganisms that the present invention can be used to assess include bacteria, filamentous fungi, and yeast. The microorganisms to be assessed can be in vegetative form or in spore form.

The bacteria to be assessed include B. atrophaeus, ATCC #9372, B. diminuta, ATCC#19146, Pseudomonas aeruginosa (ATCC #27853)

The filamentous fungi can be molds, rusts, mildews, or smuts. The filamentous fungi to be assessed include Candida, Aspergillus and Cladosporium spores.

The counting method should be suitable for the microorganisms to be assessed. For example, Tryptic soy agar plate should be used for vegetative cells of B. diminuta, ATCC#19146, while AK agar #2 plate should be used for spore suspension of B. atrophaeus, ATCC #9372. For bacteria in viable but non-culturable (VBNC) state, total cell count procedures should be adopted. (Heidelberg, et al, Appl Environ Microbiol, 63:3585-3588 (1997))

### 3. The Device Assessing Airborne Microorganisms.

The present invention provides a device for assessing the enumeration of airborne microorganisms. The device comprises a polymeric pad, which is soluble in a buffered salt solution.

The polymeric pad can be used as a settling plate, or as a filtration membrane in a commercial solid support device.

The following are some examples of the commercial solid support device: GE Osmonics Labstore manufactures polypropylene filter holders for membranes 13, 25 and 47 mm diameter filters (http://www.osmolabstore.com/OsmoLabPage.dll?BuildPage&1&1 &413); BGI Incorporated also manufactures aluminum filter holders for aerosol sampling and atmospheric pollution applications (http://www.bgiusa.com/agc/holder.htm), additional information may be found in R. A. Gussman. R. Dennis and a L. Silverman, “Notes on the Design and Leak Testing of Sampling Filter Holders”. American Industrial Hygiene Association J., 23 (November-December 1962), and finally HI-Q Environmental Products has filter holders for square flat sheets like the one shown at the link (http://www.hiq.net/Item/Page28BottomMiddle.htm) which is a CFPH-Series2 for square filter pads. There are many more supplier with capability for custom applications for filter sizing.

The filtration membrane is capable of retaining organisms suspended in air via gravitational settling or forced impact. The polymeric pad should be soluble in a buffered solution of sodium citrate. On the other hand, the polymeric pad is preferably insoluble in water, so that it can be moistened to better retain the airborne organisms. The pad is preferably porous.

The filtration pad can be made of calcium alginate. The calcium alginate pads can be placed in the environment to pick lip higher concentration of organism and further be dissolved in an appropriate buffered salt solution, such as sodium citrate, followed by serial dilution and plating. For example, the calcium alginate pad can be a Kalginate™ surgical pad for wound dressing manufactured by DeRoyal Wound Care (http://www.deroyal.com/woundcare/wcdefault.asp).

As shown in the examples, the invention was demonstrated with vegetative cells of B. diminuta, ATCC#19146, Pseudomonas aeruginosa, ATTC 27853 and spore suspension of B. atrophaeus, ATCC #9372 (formerly B. subtilus). The experiments demonstrated that calcium-alginate does not inhibit the growth of the organisms tested: B. atrophaeus and B. diminuta. Inoculation of vegetative cells and spores resulted in 100% recovery, from the polymeric membrane, indicating that this technology can be applied to collect samples in the environment of organisms such as bacteria, mold, yeast and particles.

### EXAMPLES

Examples are provided below to further illustrate different features of the present invention. The examples also illustrate useful methodology for practicing the invention. These examples do not limit the claimed invention.

### Example 1

### Viability trial of Bacillus spores

Aerosolization of Bacillus atrophaeus ATCC9372 was assessed first using a Collision Nebulizer.

B. atrophaeus spores are prepared by thawing the organism frozen vial and transferring it to Sobybeam. Casein Digest Medium. The medium is incubated for two days at 32° C. Once grown for two days, AK Agar #2 is inoculated with organism and incubated for five days at 32° C. These colonies are picked manually with a scraper and suspended in Buffered Salt solution or Distilled water. Before use, the organisms were suspended in 100 mL of distilled water at a concentration of 1×10⁵ spores/ml. The suspension is heat-shocked at 82.5° C. for 10 minutes to ensure complete sporulation before using it. This spore suspension was added to a 24 Jet Collision Nebulizer (BGI Incorporated, Waltham, Mass.) The flow rate of the Collision Nebulizer was set at ˜23.88 STD L/min and stabilization interval of 5 minutes at flow rate.

At 25.9° C. and 100.3 KPa, impingers filled with 50 mL of Buffered Salt solution were blip used to sample the organisms suspended in the air at the following flow rates.

TABLE 1

| | | Sampling | Concentration |
|---|---|---|---|
| Aerosol Sample | T_{F} | time | (CFU/m³) |
| Impinger 1 | 1.48 ± 0.4 std L/min | 10 minutes | 1.28 × 10⁶ |
| Impinger 2 | 1.39 ± 0.4 std L/min | 10 minutes | 8.629 × 10⁵ |
| Impinger 3 | 2.47 ± 0.4 std L/min | 10 minutes | 4.676 × 10⁵ |

The solution from the impingers was serially diluted and plated on Tryptic Soy Agar. The plates were incubated at 32.5° C. for 2 days and the colonies were counted. The impinger recovery 1 was enumerated as follows:

$$\frac{346 C F U \text{/} mL \times 50 mL \times 1000}{1.48 L \text{/} min \times 10 min}$$

Similarly, aerosolization of Bacillus atrophaeus ATCC9372 was also assessed using a TS1 Atomizer.

B. atrophaeus spores were suspended in distilled water at 100 ml at 1×10⁵ spores/ml. The spore suspension was added to a TSI 3079 Atomizer (TSI Incorporated, St. Paul, Minn.). The flow rate of the Atomizer was set at 200 ml/hr with no stabilization interval.

At 24.0° C. and 100.8 KPa, impinger samples were collected in 50 mls CM80 at the following flow rates.

TABLE 2

| | Flow rate | Sampling | Concentration |
|---|---|---|---|
| Aerosol Sample | (STD L/min) | time | (CFU/m³) |
| Impinger 1 | 1.45 | 30 minutes | 5.4 × 10⁴ |
| Impinger 2 | 1.45 | 30 minutes | 4.59 × 10⁴ |
| Impinger 3 | 1.26 | 30 minutes | 5.03 × 10⁴ |

### Example 2

### Viability of B. diminuta and B. subtilus in Kalginate™

The effect of Calcium Alginate pads (Kalginate™) on the viability of B. diminuta and spores of B. subtilus was determined.

A 5% solution sodium citrate (pH 9.15) was prepared. The 5% sodium citrate solution was titrated with the 5% citric acid solution, to pH 7.26.

The neutral sodium citrate solution is filter sterilized using a Millipak 20 and with peristaltic pump. The first 10 ml was discarded and remaining solution was dispensed into 50 mL aliquots into 60 mL Nalgene bottles (Nalgene). Three bottles are used for B. diminuta and three for B. subtilus.

Calcium Alginate pads (Kalginate™) was prepared using alcohol wiped scissors. The Calcium Alginate pads (Kalginate™) package was opened under the Bio-safety cabinet. The square pad was carefully removed from the package and cut into several 2×2 cm² square. Each square was placed in a 15×60 mm Petri-dish. The dishes were labeled.

A 500 μl aliquot of the B. subtilis and B. diminuta suspension was inoculated onto each Kalginate™ square. At the same time, 1.0 ml of the organism suspension was added to 9.0 ml of buffered citrate solution. This 1:10 dilution was used as control.

The inoculated squares were held inside the safety cabinet for one hour. At the end of the hold period, the squares were added to the 60 ml Nalgene bottle with the buffered citrate solution. The dissolution of the strips occurred within 2.0 minutes. A 1.0 ml of the citrate/calginate suspension was added to 9.0 ml of buffered saline solution to prepare a 1:10 dilution. Two more dilutions from the suspension were done in buffered saline.

The original concentration was about 10⁸ CFU/mL for B. diminuta, and about 10⁵ CFU/mL for B. subtilis.

All plates were incubated at 30° C. for 48 hours. Results after holding the citrate alginate suspension for 40 minutes and plating them on TSA, were as follows:

TABLE 3

| Plate Number | B. subtilus (10⁴ dilution) | B. diminuta (10⁵ dilution) |
|---|---|---|
| Plate #1 | 7.0 CFU/mL | TNTC |
| Plate #2 | 7.0 CFU/mL | TNTC |
| Plate #3 | 8.0 CFU/mL | TNTC |

The controls also were TNTC (too many to count) in a two serial dilution experiment.

### Example 3

### Calcium Alginate Filter Pad Application for Spore Recovery

An aerosol recovery assessment of the Calcium Alginate pads was performed using Bacillus atrophaeus (formerly B. subtilis) spores aerosolized with a TSI Atomizer. The outlet of the atomizer was connected to a filter holder containing calcium alginate pads. The outlet of the filter was connected to all impinger, a pre-filter and a mass flow meter. The mass flow meter was connected to the outlet of the impinger.

Sampling impingers were loaded with 50 ml of buffered salt solution. A B. atrophaeus spore suspension was prepared at a concentration of 10⁵ spores/ml. Calcium alginate dissolution buffer was 5% sodium citrate pH 7.28. During aerosolization three separate runs were performed with different filter pads:

TABLE 4

| | | Contact time in | | |
|---|---|---|---|---|
| | Aerosol | citrate buffer | TSI 3063 | |
| Trial | Emission Time | before plating | mass flow | Total volume |
| Number | (minutes) | (minutes) | (STD L/min)* | sampled (L) |
| 1 | 15 | 40 | 2.80 | 42 |
| 2 | 21 | 18 | 1.58 | 33 |
| 3 | 23 | 7 | 1.78 | 42 |

*Total sampling time of 23 minutes, except for Trial #3
**Trial #3 flow rate varied as follow: .96 std L/min (2.0 min); flow rate of 1.52 std L/min for 2.0 minutes; flow rate progressed to 1.78 std L/min for 21 minutes.
TS1 3063 mass flow 0

The enumeration results of the trial are as follows:

TABLE 5

| | Enumeration of the | Concentration of organisms in |
|---|---|---|
| Impinger | undiluted impinger | the effluent air (CFU/L) |
| 1: | 47 CFU/ml | 54 CFU/L |
| 2: | 40 CFU/ml | 46 CFU/L |
| 3: | 38 CFU/ml | 50 CFU/L |

The Kalginate™ filter pad was able to remove 99% of the organism present in the air stream based on the overall aersolization rate for the TSI Atomizer of 10⁴ CFU/m³ as shown in example #1.

### Example 4

### Calcium Alginate Pad Settling Plate Application for Snore Recovery

A spore recovery aerosol assessment was performed using calcium alginate pads employed as settling plates in a 1.0 m³ aerosol chamber. Spore suspension of B. atrophaeus at a concentration of 2.2×10⁸ CFU/ml. A Total of 6.0 ml of this suspension was aerosolized in the chamber to yield a challenge level of 1.3×10⁹ CFU/m³.

Four different impingers with 20 ml of buffered saline solution were used to collect air samples throughout the chamber ports, and Kalginate™ pads were placed inside the chamber also at seven different locations. Upon total aerosolization with the DeVilbiss® nebulizer, the aerosolized organisms inside the chamber were allowed to settle for one hour. Enumeration was performed with the impinger solution and the Kalginate™ pads were dissolved in the appropriate sodium citrate buffer, diluted and plated.

TABLE 6

| | | Sample | | |
|---|---|---|---|---|
| | Flowrate | collection | Volume of | Organism |
| Impinger | (STD | time | air sampled | enumeration in the |
| Number | L/min) | (minutes) | (L) | chamber air (CFU/L) |
| 1 | 4.92 | 2.0 | 9.84 | 1.26 × 10⁵ |
| 2 | 4.92 | 2.0 | 9.84 | 1.16 × 10⁵ |
| 3 | 4.89 | 2.0 | 9.78 | 1.30 × 10⁵ |
| 4 | 4.86 | 2.0 | 9.72 | 6.99 × 10⁴ |

The Kalginate™ pads were located two settling plates front-back of the cabinet (left and right sides), two center (front and back) and one in the middle. The total deposition time for the organism suspended in the air was one hour. The Kalginate™ pads were removed from the chamber, dissolved in 5% sodium citrate buffered, serially dilated and then plated in 3M Petri-film™ aerobic count plates.

TABLE 7

| Chamber Location | Enumeration (CFU/cm²) |
|---|---|
| Front right | 2.7 × 10⁴ |
| Front center | 3.1 × 10⁴ |
| Front Left | 3.9 × 10⁴ |
| Center | 3.2 × 10⁴ |
| Back right | 2.2 × 10⁴ |
| Back center | 3.6 × 10⁴ |
| Back left | 2.9 × 10⁴ |

Based on the amount of spores input inside the chamber, that is 1.3.2×10⁹ CFU in 1.0 m³ of total chamber volume, the average enumeration of the surface sampled with Kalginate™ pads is 3.08×10⁴ CFU/m² or 3.08×10⁸ CFU/m². The average enumeration converted to volumetric values can be done by assuming that the chamber is a perfect cube. Since a cube has 6 sides, the total organism enumeration in the chamber is approximately 1.84×10⁹ CFU in 1.0 m³. Taking into account the dilution, enumeration errors, the Kalginate™ pads are able to determine the concentration of organisms in the chamber more accurately than impingers.

Other embodiments are within the following claims. While several embodiments have been shown and described, various modifications may be made without departing from the spirit and scope of the present invention.

What is claimed is:

1. An environmental sampling method for assessing airborne microorganisms comprising,
retaining organisms suspended in air to a polymeric pad via gravitational settling or forced impact,
dissolving the polymeric pad in a buffered salt solution, and
determining the number and kind of microorganisms in the buffered salt solution;
wherein neither the polymeric pad nor the buffered salt solution inhibits the growth of the microorganisms to be determined.

2. The method of claim 1 wherein the polymeric pad is porous.

3. The method of claim 2 wherein the polymeric pad is a calcium alginate pad.

4. The method of claim 3 wherein the buffered salt solution is a buffered sodium solution.

5. The method of claim 4 wherein the buffered sodium solution is a sodium citrate solution with the pH at about 7.

6. The method of claim 1 wherein the microorganisms to be determined are selected from the group consisting of bacteria, yeast, and filamentous fungi.

7. The method of claim 6 wherein the microorganisms comprise B. diminuta.

8. The method of claim 6 wherein the microorganisms comprise P. aeruginosa.

9. The method of claim 6 wherein the microorganisms comprise B. atrophaeus.

10. The method of claim 1 wherein the step of determining is achieved by diluting the buffer, pouring the diluted buffer solution on growth media, incubating the plates in the conditions suitable for the growth of the organisms to be determined, and counting the colonies.
